(19) United States
(12) Patent Application Publication
(10) Pub. No.: US 2020/0405535 A1
(43) Pub. Date: Dec. 31, 2020

(54) COOLING PAD; COOLING APPARATUS; COOLING SYSTEM AND METHOD FOR OPERATING A COOLING PAD AND A COOLING APPARATUS

(71) Applicant: NMI Naturwissenschaftliches und Medizinisches Institut an der Universitaet Tuebingen, Reutlingen (DE)

(72) Inventors: Martin Stelzle, Reutlingen (DE); Gorden Steve Link, Tuebingen (DE); Simon Werner, Gomaringen (DE); Britta Hagmeyer, Tuebingen (DE)

(21) Appl. No.: 17/018,704

(22) Filed: Sep. 11, 2020

Related U.S. Application Data
(63) Continuation of application No. PCT/EP2019/056032, filed on Mar. 11, 2019.

(30) Foreign Application Priority Data
Mar. 12, 2018 (DE) 102018105674.5

(51) Int. Cl. A61F 7/10; F04B 43/12; F04B 51/00; F04C 2/08

(52) U.S. Cl. CPC A61F 7/10; F04B 43/12; A61F 2007/0056; F04C 2/08; F04B 51/00

(57) ABSTRACT

A cooling pad (10) for medical or veterinary applications, the cooling pad comprising a first heat exchanger (20); a second heat exchanger (30), a heat transfer fluid; a piping system (40); and an integrated pump device (50) for the heat transfer fluid; wherein the first heat exchanger (20) and the second heat exchanger (30) have the heat transfer fluid flowing through them; wherein the piping system (40) fluidically connects the first heat exchanger and the second heat exchanger; wherein the pump device (50) is adapted to cause an exchange of the heat transfer fluid between the first heat exchanger and the second heat exchanger; wherein the cooling pad (10) comprises a hermetically sealed fluid circuit for the heat transfer fluid, the fluid circuit comprising the first heat exchanger (20), the second heat exchanger (30), the piping system (40) and the pump device (50). Further, a corresponding cooling device (60), cooling system (1) and corresponding method (200) are proposed.

### CROSS REFERENCE TO RELATED APPLICATIONS

This is a continuation application of co-pending international patent application PCT/EP2019/056032, filed Mar. 11, 2019 and designating the United States, which was published in German as WO 2019/175111 A1, and claims priority to German patent application DE 10 2018 105 674.5, filed Mar. 12, 2018, both of which are incorporated herein by reference.

### FIELD

The present disclosure relates to the field of medical technology and in particular to a cooling bandage or cooling pad, in particular for applications in human and veterinary medicine, a corresponding cooling device, as well as a method for operating the cooling pad and cooling device.

### BACKGROUND

Cooling pads or cooling bandages and cooling devices are generally known from the prior art.

In Germany, about 1.5 million people get injured in sports accidents every year. For initial treatment of acute injuries and for post-operative treatment, the most important measure is cooling treatment. Furthermore, cooling extremities, e.g. hands and feet, during and after chemotherapy can prevent damage to tissue and peripheral nerves.

The effect of cooling on the skin and the underlying connective tissue leads on the one hand to a narrowing of the blood vessels (vasoconstriction) and a reduction of the local blood circulation. On the other hand, the cell metabolism is slowed down and the activity of proinflammatory substances is inhibited and the transmission of pain is delayed. Cooling therapy thus causes rapid pain relief and local de-swelling of the injured tissue. The resulting faster healing process shortens the rehabilitation period and reduces the disease-related costs that may arise from loss of working hours and hospitalization.

Currently, cold wraps, ice bags, pre-cooled thermo-packs containing gel, cold compresses (cooling due to endothermic chemical reaction) or ice/cold spray are mainly used for cold therapy or cryotherapy.

One disadvantage of these solutions, however, is that if they are not used properly, they often cool too much, causing cold damage and even localized frostbite in the tissue. Cold damage sometimes has the opposite effect of delaying rather than promoting healing.

A further disadvantage can be that the application for long-term aftercare of injuries in everyday life is very costly, as the cooling products warm up after a short time and have to be replaced again. A safe, long-lasting and consistent, appropriate moderate cooling effect is in many cases not achieved successfully.

U.S. Pat. No. 4,962,761 discloses an improved cooling system with controlled temperature regulation. The system includes a fluid circulation and temperature control device to which different cooling pads can be connected. Depending on the application, different cooling pads can be connected to the coolant circuit.

A disadvantage of this solution, however, is that the handling is complex. If not handled properly, the coolant can leak, especially when the user fills the coolant or when connecting it to the coolant circuit.

EP 0 039 443 A1 discloses a device for cooling body portions or parts of the body in the context of therapeutic or diagnostic measures. The device essentially consists of a wound pad or bandage through which a heat transport medium flows, a cooling unit and measuring and control devices for controlling the heat transport. The bandage is connected to the supply unit or cooling unit by means of hoses and fluid couplings that can be closed on both sides. The risk of leakage is reduced in that the connection to the hoses of the bandage is made by self-locking fluid couplings. In other words, a kind of non-return valve is provided during coolant transfer.

Yet a different approach is disclosed in DE 42 19 392 A1. Therein an orthopedic support bandage is described for supporting a healing processes in the area of joints with stiff support inserts to relieve the joint. No coolant or heat transfer medium is provided. Instead, temperature-controlled electrical heating and/or cooling elements are proposed which are arranged directly inside of the support bandage, in particular resistive heating foils and/or Peltier elements. A disadvantage of Peltier elements, however, is that Peltier elements are not mechanically flexible and therefore cannot be used directly in an orthosis that is adapted to the body shape. It can be very difficult to find a comfortable wearing position that provides targeted cooling.

### SUMMARY

Against this background, it would among other objects be desirable to provide a further improved cooling pad and cooling device wherein the handling can be improved. In particular, it would be desirable to provide an improved combination of cooling pad and cooling device, which may eliminate disadvantages of the aforementioned cooling devices and may combine advantages thereof.

According to a first aspect of the present disclosure, a (fluid-filled) cooling pad is provided in particular for medical or veterinary applications, the cooling pad comprising:
a first heat exchanger;
a second heat exchanger,
a heat transfer fluid;
a piping system; and
an integrated pump device (circulating pump) for the heat transfer fluid;
wherein the first heat exchanger and the second heat exchanger have the heat transfer fluid flowing through them;
whereby the piping system connects the first heat exchanger fluidically with the second heat exchanger;
wherein the pump device is adapted to cause an exchange of the heat transfer fluid between the first heat exchanger and the second heat exchanger;
wherein the cooling pad comprises a hermetically sealed fluid circuit for the heat transfer fluid, wherein the hermetically sealed fluid circuit comprises the first heat exchanger, the second heat exchanger, the piping system and the pump device.

According to a second aspect of the present disclosure, a corresponding cooling device, in particular for medical or veterinary applications, is presented, the cooling device comprising:
a receptacle adapted to accommodate a heat exchanger of a cooling pad (as described above);
a cooling element, in particular comprising a thermoelectric cooling element such as a Peltier element; wherein the cooling element is arranged to supply heat to the heat exchanger of the cooling pad or to remove heat therefrom when the heat exchanger of the cooling pad is placed in the receptacle. The cooling device may further comprise a drive adapted to drive a pump device integrated in the cooling pad.

According to another aspect of the present disclosure, a cooling system is presented, the cooling system comprising a cooling pad and a corresponding cooling device as mentioned above.

According to another aspect of the present disclosure, a method for operating a cooling pad and a cooling device is presented, the method comprising the steps of:
providing the above mentioned cooling pad and the corresponding cooling device.
placing the second heat exchanger of the cooling pad into a receptacle of the cooling device;
application of the first heat exchanger of the cooling pad to a location to be cooled;
starting the cooling process with the cooling device.

It shall be understood that the cooling device, cooling system and method may have similar or identical respective embodiments as described below in detail for the cooling pad according to the present disclosure.

In legacy fluid-based solutions, there is a fluid exchange between a cooling device and a cooling pad. However, the handling of such “wet” cooling systems is problematic. Especially with untrained personnel, coolant leakage can occur.

In contrast thereto, the cooling pad proposed by the inventors features a (intrinsically) hermetically sealed fluid circuit. Instead of requiring a fluid interface to an external supply unit, a second heat exchanger is provided. Via the second heat exchanger, heat absorbed by the first heat exchanger can be transferred to a cooling device. Consequently, the proposed cooling device does not require a fluid interface. Instead, a receptacle for accommodating the second heat exchanger is provided. The cooling element of the cooling device adapted accordingly to dissipate heat from the accommodated heat exchanger of the proposed cooling pad, when placed in the receptacle. This results in the desired heat exchange, but without the user having to manipulate the fluid circuit. Hence, there is no need to exchange a coolant or heat transfer fluid between the cooling pad and the cooling device.

Accordingly, a fully integrated, closed, fluid-filled cooling pad is advantageously provided. The fluid circuit is completely closed and encapsulated in the cooling pad itself. An advantage of a pre-filled cooling pad with hermetically sealed fluid circuit is the simple, uncomplicated handling. It is not necessary, that the user deals with a coolant. In particular, the proposed cooling pad does not have a fluid interface to be operated by a user. A leakage of coolant due to improper handling can thus be avoided. In conventional fluid-based cooling pads this is problematic when establishing connection of the coolant circuit. In order to prevent leakage, the prior art merely suggests non-return valves. However, this is associated with additional cost and effort.

Another advantage of the proposed solution over conventional fluid-based cooling systems may also be that hygiene can be improved. Because the proposed cooling pad can be prefilled, there is no need for the user to handle a coolant near the patient, possibly in an area of an open wound. Furthermore, thanks to the closed fluid circuit, the risk of cross-contamination between different patients using the same cooling pad, for example, one after the other, can be avoided.

Accordingly, an advantage of the proposed cooling device can be that the cooling device only accommodates the heat exchanger but there is no direct fluid contact.

Another advantage of the proposed solution can be the simple and quick exchange of the cooling pads. Besides quality improvements, the efficiency of patient care can thus also be increased.

A further advantage may be that a low thermal inertia can be achieved by an optimized, preferably very small fluid volume. This enables rapid cooling and, due to the low heat capacity, low power consumption. Consequently, a smaller, lighter system with improved mobility is possible. Due to the spatial separation of cooling device and cooling pad, there is preferably no backflow of heat and undesired warming of the tissue may be avoided.

An advantage over the above-mentioned DE 42 19 392 A1, in which an electrical heating and/or cooling element is arranged directly inside a bandage (without fluid circuit), can be that the first and second heat exchangers can be arranged at a distance from each other. This allows heat to be moved away from the body part to be cooled. When used as a cooling bandage, DE 42 19 392 A1, on the other hand, does not provide a solution for the problem of removing heat. If a Peltier element is used, the sum of the thermal power dissipated by the tissue and the electrical power supplied would have to be dissipated via some kind of heat sink to the environment. Hence, especially under clothing or under an existing orthosis, there is limited use. Hence, the solution shown in DE 42 19 392 A1 may in fact only be useful as a thermal bandage.

Compared to conventional pre-cooled gel packs, an advantage can be that no existing cooling infrastructure, such as refrigerators or freezers, is required. In particular, the proposed cooling device may have an energy storage device, such as a battery or accumulator, and can therefore be operated away from an existing cooling infrastructure.

In the context of the present disclosure, the term cooling or cold treatment can generally refer to thermal treatment or inducing a change in temperature. This may include both the supply and removal of heat. Consequently, a cooling pad may also be used for heating besides cooling. The same applies to a cooling device, cooling system or method. For easier understanding, in the context of the present disclosure reference is made to cooling in a shortened form, but without limitation.

A pump device can be understood as a circulating pump for the heat transfer fluid. The drive unit of the pump device can be understood as a separate entity, which is preferably part of the cooling device interacting with the cooling pad.

Advantageously, the first or the second heat exchanger may comprise a heat absorption area for absorbing heat and the other heat exchanger may comprise a heat release or heat emission area for releasing heat. The cooling pad with its hermetically sealed fluid circuit for the heat transfer fluid is adapted to transfer heat between the first heat exchanger and the second heat exchanger. The piping system serves for heat transfer between the first and second heat exchanger, and thus for heat transfer between the body region to be cooled and the cooling device. For example, the first heat exchanger absorbs heat, the heat is transported to the second heat exchanger by means of pump device and piping system, and there it is released, e.g. to a cooling device. The proposed cooling pad is thus an intermediate piece between the body region to be cooled and a remote cooling device, but without requiring fluid coupling to the cooling device.

Advantageously, the piping system of the cooling pad may comprise a flow line and a return line, wherein the flow line fluidly connects an inlet or input of the first heat exchanger and an outlet or outlet of the second heat exchanger; and wherein the return line fluidly connects an outlet or output of the first heat exchanger and an inlet or input of the second heat exchanger. A continuous circulation can thus advantageously be enabled.

Advantageously, the pump device can be adapted to be driven by an external drive without any opening or lead-through. In other words, the pump device advantageously has no additional opening for the drive equipment. This can reduce the probability of leakage of the heat transfer fluid. An advantage of this embodiment can be that the cooling pad with its hermetically closed fluid circuit is even better sealed off from the outside world. In particular, the pumping equipment does not have any mechanical, electrical and/or fluidic feed-through. For example, no mechanical feed-through is provided for a drive shaft of the pump device.

In a refinement, the pump device can be adapted to be magnetically coupled to an external drive unit. In particular, the pump device can be a magnetically coupled gear type pump. The force transmission can thus be effected magnetically without a feed-through. Advantages of this embodiment can be a high energy efficiency and increased safety. A direct connection to the drive or contact to movable parts of the pump device is not necessary. It is to be understood that other pump types, such as a centrifugal pump or rotary lobe pump, can also be used. Optionally, the pump device can be equipped with an eddy current drive. The pump device can therefore be adapted to be driven by eddy current. In particular, the pump device can be driven by an eddy-current drive unit without the need for a feedthrough. For example, the pump device can be a gear type pump adapted to be driven by eddy current. For example, a gear wheel of the gear type pump may comprise an element, such as a disc, in which an eddy current can be induced. For example, a gear wheel may be made of a metal such as copper. Advantageously, corrosion protection may also be provided, such as a coating for a copper gear.

The pump device can be a peristaltic pump. A drive unit can act peristaltically on the pump device from the outside and thus cause an transfer of the heat transfer fluid between the first heat exchanger and the second heat exchanger. Peristaltic pumps are based on the principle of displacement by squeezing sections, here for example squeezing of the piping system or the first and/or second heat exchanger or a pipe included in the cooling pad. An advantage of this embodiment can be a very cost-effective and still lead-through-free design.

The pump device may comprise a diaphragm pump. A diaphragm pump may comprise a chamber covered by a diaphragm and an inlet and an outlet. Inlet and outlet can be closed by one-way valves in such a way that when the diaphragm is moved (e.g. up and down movement) fluid is transported from the inlet to the outlet.

Advantageously, at least a portion of the first heat exchanger is mechanically flexible. An advantage of this embodiment may be that the cooling pad can be flexibly adapted to a shape of an area to be cooled, for example a body shape or a fixture within an orthosis. Another advantage can be that the same cooling pad can be used in different application scenarios, thus reducing inventory.

Advantageously, the first heat exchanger can be adapted to a body part to be cooled. The adaptation can be done in many ways, in particular regarding the shape, the length of the piping system and/or adapted to a required heat transfer capacity. Furthermore, for example, an overall height can be adapted in such a way that the first heat exchanger can be integrated into an existing orthosis or worn underneath it.

Advantageously, the cooling pad can have a diffusion-inhibiting coating. The coating can advantageously inhibit or reduce diffusion of the heat transfer fluid. For example, the coating is water vapor-tight. An advantage of this embodiment is that the shelf life of prefilled cooling pads can be improved. For example, the coating comprises at least one of a (thin) metal layer, a silicon oxide layer or multi-layers of metal or inorganic thin layers.

Advantageously, the cooling pad may also comprise a fluid reservoir adapted to compensate for fluid losses of the heat transfer fluid. An advantage of this embodiment can be that the functionality can be ensured even over longer storage periods. The reservoir can optionally be adapted as an overpressure reservoir. Optionally, the reservoir can be adapted such that it is pressurized during its intended use in a cooling device, for example by a corresponding spring mechanism of the cooling device. Advantageously, the reservoir can be thermally decoupled. For example, the reservoir can be integrated into the fluid circuit via a T-piece. Advantageously, the reservoir is adapted in such a way that a pressure loss can be compensated, but does not directly contribute to the heat capacity of the fluid circuit, which should advantageously be kept low.

Advantageously, the first and/or second heat exchanger can have bifilarly arranged cooling loops or bifilarly arranged heat transfer fluid carrying heat exchanger channels. An advantage of this embodiment may be that a more even temperature distribution on the skin can be achieved. The cooling loops can, for example, be arranged in a meandering pattern. At least two adjacent cooling loops can always have the same average distance from a transition of the first heat exchanger to the piping system.

Advantageously, fluid channels carrying heat transfer fluid in at least one of the first heat exchanger and the second heat exchanger may have a semi-circular channel cross-section. The fluid channels can be arranged in such a way that the flattened side of the semicircular channel cross-section faces a side of the first and/or second heat exchanger via which heat is to be absorbed or dissipated. An advantage of this embodiment can be that the largest possible contact surface to the body can be achieved with minimum/low flow resistance and/or minimum/low total volume of the heat transfer fluid.

Advantageously, the first heat exchanger can have a thickness between 1 mm and 10 mm, in particular between 2 mm and 8 mm, in particular between 4 mm and 6 mm. An advantage of this embodiment may be that it can be placed underneath an existing orthosis.

Advantageously, the first heat exchanger may comprise a heat exchanger surface and cooling loops arranged within the heat exchanger and wherein the cooling loops or heat transfer fluid are separated from the heat exchange surface by a thin, thermally conductive and fluid-tight membrane. An advantage of this embodiment can be an efficient heat transfer between a location to be cooled and the cooling pad. Advantageously the membrane has a thickness of no more than 250 μm, in particular no more than 100 μm.

Advantageously, the cooling pad may comprise at least in parts a biocompatible, thermally conductive, in particular as thin as possible, open-pored fabric layer, in particular in a region of a surface of the first heat exchanger. This can improve the wearing comfort even with direct skin contact and may prevents skin irritation, while at the same time providing high mechanical stability thanks to the fabric layer.

Advantageously, fluid channels in at least one of the first heat exchanger, the second heat exchanger and the piping system may have a diameter between 2 mm and 3 mm. An advantage of this embodiment is that good heat transfer properties, low thickness of the cooling pad and a not too high fluid resistance are combined in an advantageous way.

Advantageously, the piping system between the first heat exchanger and the second heat exchanger may have a length between 2 cm and 300 cm, in particular between 5 cm and 100 cm, in particular between 10 cm or 20 cm and 50 cm. Advantages of this embodiment can be that the first and second heat exchangers can be easily positioned spatially separated, while at the same time the volume of fluid in the fluid circuit remains low.

Advantageously, the fluid circuit may have a fluid volume between 5 ml and 100 ml, in particular between 10 ml and 60 ml, in particular between 15 ml and 40 ml, in particular between 20 ml and 30 ml. An advantage of this embodiment is a fast cooling effect. By keeping the heat capacity of the heat transfer fluid in the fluid circuit low, the system enables improved heat transfer, so that a high cooling rate e.g. cooling rates of approx. 5-10 K/min can be achieved. A fast response behavior is thus provided. A further advantage can be that efficient battery-powered operation of the cooling device becomes possible, as less cooling power is required. Furthermore, the treatment and wearing comfort may be improved. In particular, a reduced weight at the treatment position can be provided. Optionally, the fluid circuit can have a fluid volume of 0.01-0.2 ml per cm² of cooling surface or heat exchanger surface +, if necessary, an offset of 10-50 ml for the fluid volume of the supply line.

Advantageously, the heat transfer fluid may comprise water and/or ethylene glycol. A coolant with a high heat capacity is advantageous, since only a small volume of liquid or a low flow rate is required to effectively dissipate a sufficient amount of heat. Water, water/ethylene glycol or similar can be used as heat transfer medium or coolant. Because the fluid circuit is hermetically sealed, a wide variety of coolants can be used. Even in the case of improper handling, there is no risk of the coolant coming into contact with the patient or even an open wound to be protected, such that patient safety is improved. Furthermore, inexpensive coolants may be used.

Advantageously the cooling pad can be set up for a heat flow between 15 mW and 100 mW, in particular between 15 mW and 80 mW, in particular between 25 mW and 65 mW per square centimeter of surface area of the first heat exchanger. The inventors have recognized that such heat flows enable a particularly advantageous cooling effect. The measurement of a temperature dependent heat flow rate is described in Link G., Stelzle, M. “Measurement of temperature dependent heat flow rate from human limbs towards thermoelectronic coolding device”, Med. Devices Diagn. Eng., Vol. 2(1): 72-74; 2017.

Advantageously, the first heat exchanger and the piping system can have a common layer structure. In particular, the first heat exchanger, the piping system and also the second heat exchanger can have a common layer structure. The common layer structure may comprise the following:
an upper cover layer,
a lower cover layer, and
an intermediate layer, in particular a foam layer, which is arranged between the upper cover layer and the lower cover layer; and wherein fluid channels of the first heat exchanger and the piping system, and in particular also of the second heat exchanger, are arranged in the intermediate layer. An advantage of this embodiment can be that it is inexpensive to manufacture. In particular, the cooling pad including the piping system and heat exchangers can be manufactured by means of laminating technology or foil technology. Thanks to the hermetically closed fluid circuit, it is not necessary to provide e.g. fluid couplings with one-way valves, which would result in costly manufacturing.

Optionally, a cooling pad for human or veterinary medicine applications can be provided, the cooling pad comprising a first heat exchanger and a piping system, in particular for connecting the first heat exchanger to a cooling device, wherein the first heat exchanger and the piping system have a common layer structure comprising an upper cover layer, a lower cover layer, and an intermediate layer, in particular a foam layer, which is arranged between the upper cover layer and the lower cover layer; and wherein fluid channels of the first heat exchanger and the piping system are arranged inside the intermediate layer. In this case, the second heat exchanger, the heat transfer fluid and the integrated pump device are optional features which may be provided but do not have to be provided. An advantage of this embodiment can be a cost effective production of the cooling pad. Such a cooling pad can be further refined according to the additional features of the present disclosure. In particular, the first heat exchanger, the piping system and also the second heat exchanger may have a common layer structure.

In a further refinement, the integrated pump device can also be arranged between the (common) upper cover layer and the (common) lower cover layer. This can further simplify the manufacturing process. In particular, a tightness of the entire system can be ensured by a surrounding coating, so that connections between heat exchangers, piping system and/or pump device can be manufactured easily and cost-effectively.

Advantageously, the cooling device according to the second aspect can further comprise a temperature sensor and a controller; wherein the temperature sensor is configured to detect a temperature of a cooling pad inserted into the receptacle and wherein the controller is configured to control the drive device and/or the cooling element based on (a setpoint value and) a temperature detected by the temperature sensor. Advantageously, control can be effected as a function of a setpoint temperature and/or a flow temperature and/or return temperature measured at the cooling pad. Alternatively or additionally, the cooling pad itself may comprise one or more temperature sensors and the controller can be set up to control the drive unit and/or the cooling element as a function of these measured values. Optionally, the cooling device can have a cooling device comprising a Peltier element. Optionally, the temperature of the Peltier element can be measured and taken into account in the control. Optionally, the cooling device may comprise a fan which can be controlled based on the temperature of the Peltier element.

Advantageously, the controller can be configured to adjust a setpoint temperature between 15 and 25° C. Optionally, the controller can be adapted to provide a preset temperature curve. For example, the controller can be configured to provide cooling down by 10 to 20 K with a cooling duration of 10 to 15 minutes and to apply this during the first 1 to 5 days after an injury or surgery several times a day.

Optionally, the cooling pad and/or the cooling device can include a telemonitoring device and/or a remote control device. An advantage of these embodiments is that the cooling application can be monitored and adjusted if necessary.

Advantageously, the cooling device may also include a monitoring device. The monitoring device can be configured to compare a (rotational) speed of the drive device with a power consumption of the drive device. Optionally, an error message can be output in case of a deviation. For example, the power consumption drops if the pump is in idle, for example if the cooling pad leaks or is not sufficiently filled, or if the drive unit does not drive the pump, for example because the cooling pad is not or not correctly inserted. Optionally, the monitoring device can thus be adapted to alternatively or additionally perform a coupling test of the pump device with the drive unit. Optionally, an optical or magnetic speed measurement of the pump device can be provided for flow rate measurement and/or control.

Advantageously the monitoring device can be configured to detect air bubbles in the cooling pad. The monitoring device may for this purpose be implemented as an optical, capacitive and/or acoustic measuring device. An advantage of this embodiment is that the detection of leaks and/or underfilling can be improved. A monitoring device can protect the pump device from idling and/or can be used for a controlled use of the above mentioned fluid reservoir.

Advantageously, the cooling device may comprise an energy storage, such as a battery or accumulator. An advantage of this embodiment is that the cooling can take place independent of a cooling infrastructure.

Further advantages are apparent from the description and the appended drawings.

It will be appreciated that the features specified above and those still to be elucidated hereinafter can be used not only in the particular combination indicated, but also in other combinations or on their own, without departing from the scope of the present invention.

### BRIEF DESCRIPTION OF THE DRAWINGS

Embodiments of the disclosure are shown in the attached drawings and will be explained in more detail in the description below.

FIG. 1 shows a schematic figure of a cooling pad according to an embodiment;

FIG. 2 shows a schematic figure of a cooling system comprising a cooling pad and an opened cooling device according to another embodiment;

FIG. 3 shows a schematic figure of a cooling system comprising a cooling pad, a closed cooling device and an orthosis;

FIG. 4 shows a schematic figure of an application of the system from FIG. 3;

FIG. 5 shows a sectional view along A-A in FIG. 1;

FIG. 6 shows a sectional view along B-B in FIG. 1;

FIG. 7 shows a schematic figure of a cooling device;

FIGS. 8 and 9 show schematic figures of a pump device of a cooling pad and a corresponding drive of a cooling device;

FIG. 10 shows a perspective schematic figure of the pump device from FIG. 9; and

FIG. 11 shows a flow chart of a method for operating a cooling pad and a cooling device.

### DESCRIPTION OF PREFERRED EMBODIMENTS

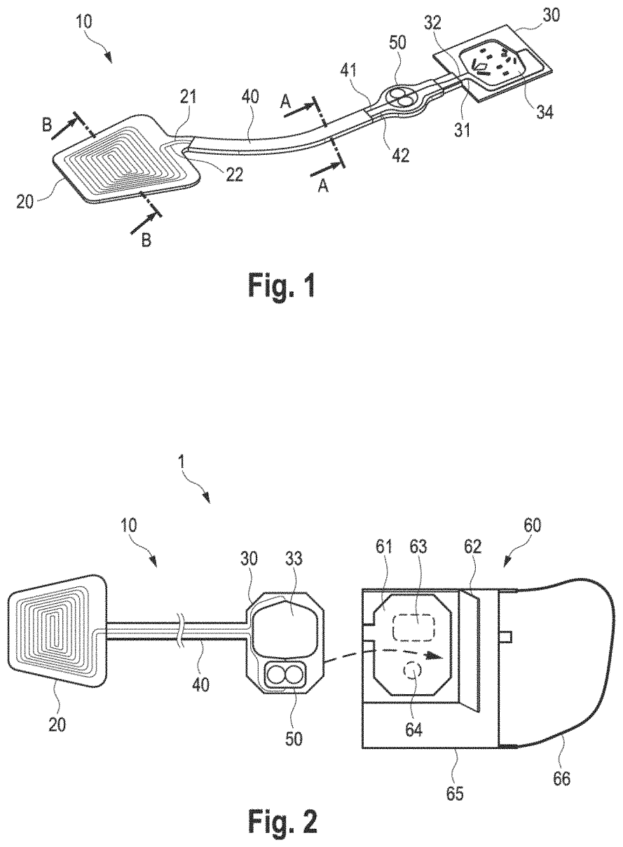

FIG. 1 shows a schematic figure of an embodiment of a cooling pad 10. The cooling pad may be used in particular for medical or veterinary applications for heat and/or cold therapy. The cooling pad 10 comprises a first heat exchanger 20 and a second heat exchanger 30. The first heat exchanger 20 and the second heat exchanger 30 are arranged at a distance from each other and are fluidically connected to each other via a piping system 40. The cooling pad 10 further comprises a pump device 50 which is adapted to cause an exchange of a heat transfer fluid between the first heat exchanger 20 and the second heat exchanger 30 via the piping system 40.

In the shown embodiment, the piping system 40 has two hose lines, a flow line 41 and a return line 42. The flow line 41 is fluidically connected to an inlet 21 of the first heat exchanger 20 and an outlet 32 of the second heat exchanger 30. The return 42 is fluidically connected to an outlet 22 of the first heat exchanger 20 and an inlet 31 of the second heat exchanger 30.

The second heat exchanger 30 may comprise flow guiding structures 34, which are adapted to create a uniform flow over the surface of the heat exchanger. Thus, the heat exchange can take place over a large part of the surface, in particular over the entire surface of the heat exchanger and no dead water with low heat exchanger efficiency may occur.

In the embodiment shown in FIG. 1, the pump device 50 is arranged between the piping system 40 and the second heat exchanger 30. However, the pump device can also be positioned at other locations, for example as shown in FIG. 2. There the pump device 50 is integrated in the second heat exchanger 30.

As can be seen from FIGS. 1 and 2, the cooling pad 10 is a fully integrated, closed, fluid-filled cooling pad. The cooling pad 10 has a hermetically sealed fluid circuit. It is therefore not necessary to connect the cooling pad to an external fluid circuit. Handling can therefore be significantly simplified.

FIG. 2 shows a schematic figure of a cooling system 1 comprising a cooling pad 10 and a cooling device 60. The cooling device comprises a receptacle 61 to accommodate the heat exchanger 30 of cooling pad 10. The cooling device may comprise a cover 62 for the receptacle 61. The cover lid 62 is illustrated opened in FIG. 2 and provides a view of the inside of the receptacle 61. There, a cooling element 63, in particular comprising a thermoelectric cooling element such as a Peltier element, and a drive 64 can be arranged.

The cooling element 63 is adapted to supply heat to or remove heat from the heat exchanger 30 of the cooling pad 10 when the heat exchanger 30 of the cooling pad is in the receptacle 61. For this purpose, the heat exchanger 30 and the cooling element 63 are advantageously arranged such that they are then position one above the other. In particular, the second heat exchanger 30 can have a areal heat dissipation region 33, which corresponds to a position of the cooling element 63.

Advantageously, the cooling element 63 or a receptacle 61 of the cooling device may be structured, for example to i) increase the heat exchanger surface and/or ii) influence a fluid flow in the heat exchanger 30 when the heat exchanger is pressed on by the cover 62. For example, flow guiding structures may be provided which can influence a flow within the accommodated heat exchanger. Advantageously, in addition or in the alternative to the flow guiding structures of the second heat exchanger 30 shown in FIG. 1, the cooling device comprise guiding structures which are adapted to cause an even flow over the surface of the heat exchanger. Thus the heat exchange can take place over a large part of the surface, advantageously over the entire surface of the heat exchanger, and no dead water with low heat exchanger efficiency may be present. An advantage of this embodiment is that the heat conducting structures are provided in the reusable cooling device and the manufacturing costs of the cooling pad can be reduced.

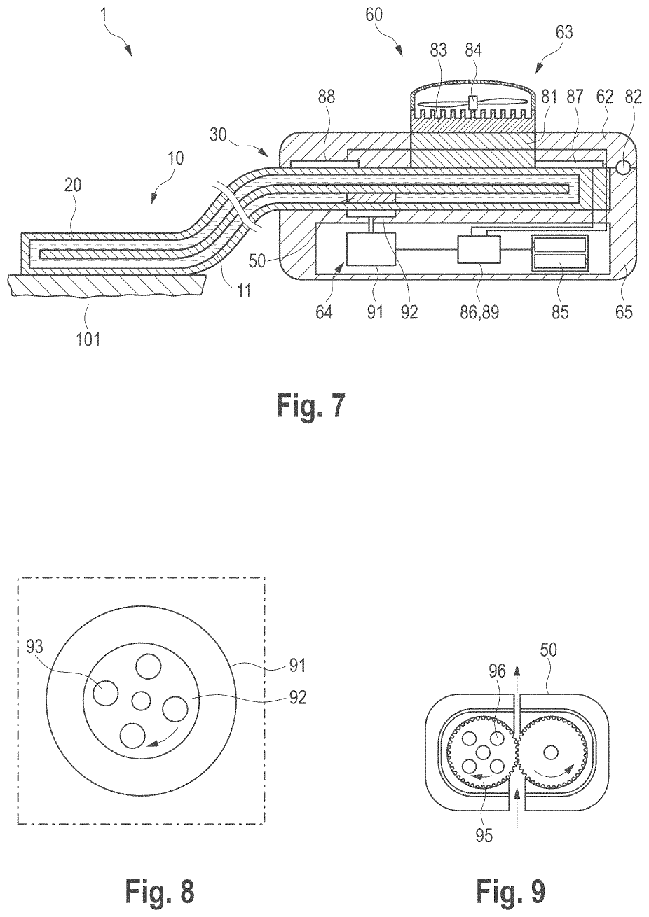

The drive 64 is adapted to drive the pump device 50 integrated in the cooling pad 10. An example of this will be explained in more detail below with reference to FIGS. 6 and 7.

The cooling device 60 may also comprise a housing 65 and a carrying strap 66.

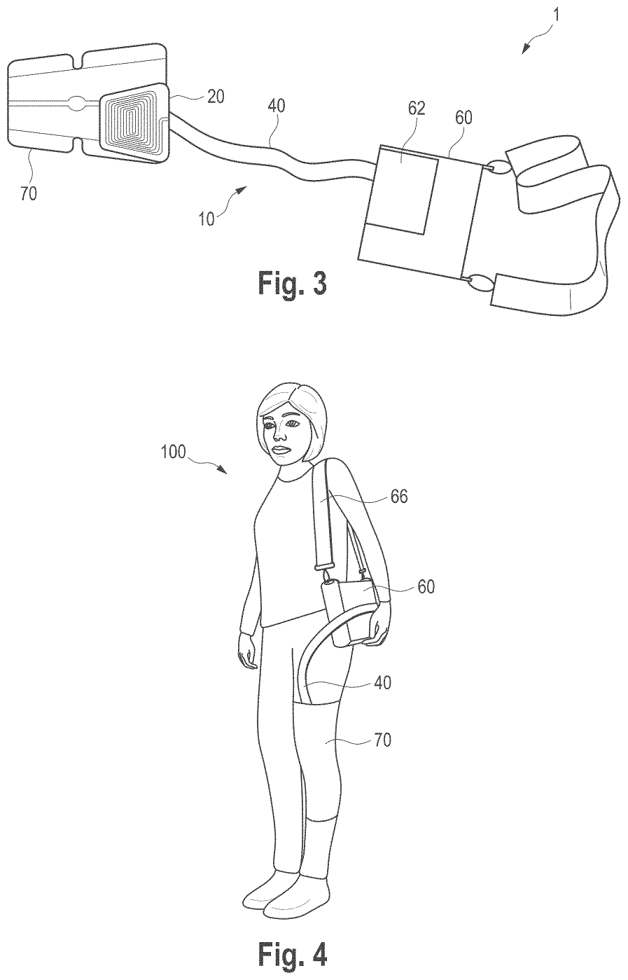

FIG. 3 shows a schematic figure of a cooling system 1 comprising a cooling pad 10, a closed cooling device 60 and additionally an orthosis 70. The second heat exchanger and the pump device 50 are accommodated in the receptacle 61 and covered by the cover lid 62. The cover lid 62 or the housing 65 therefore advantageously protects the pump device 50 and the second heat exchanger. The orthosis 70 can be a conventional orthosis.

FIG. 4 shows a user 100 with an exemplary knee orthosis 70. A thickness or height of the first heat exchanger 20 is advantageously adapted such that the heat exchanger can be worn under an orthosis 70. As shown in FIG. 4, the piping system 40 thus leads from the first heat exchanger 20 worn under the orthosis 70 to the cooling device 60. The cooling device 60 is advantageously a battery-powered cooling device that can be used as a mobile unit and independently of other cooling infrastructure. For example, the cooling device 60 can be worn with a shoulder strap 66. Alternatively, depending on the required cooling capacity, smaller units can also be provided, which can for example be worn on the belt or directly on the clothing or body.

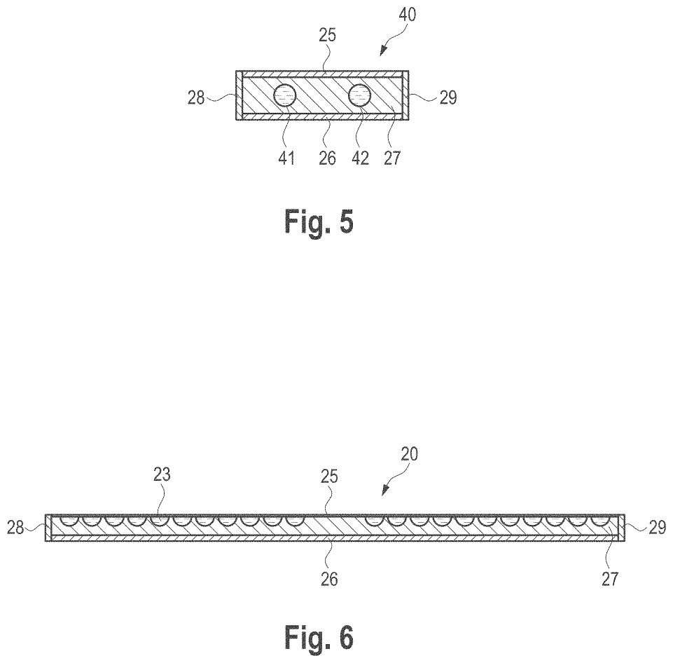

FIG. 5 and FIG. 6 show sectional views along A-A and B-B in FIG. 1, respectively. The first heat exchanger 20 (see FIG. 6) and the piping system 40 (see FIG. 5) can have a common layer structure with an upper cover layer 25 and a lower cover layer 26. Between the upper cover layer 25 and the lower cover layer 26 there is an intermediate layer 27. The fluid channels of the first heat exchanger 20 and the piping system 40 can be arranged in the intermediate layer 27. Advantageously, the second heat exchanger 40 also has a corresponding layer structure. Fluid channels of the second heat exchanger can also be arranged between the upper and lower cover layers 25, 26. Advantageously, the pump device 50 can also be arranged between the upper cover layer 25 and the lower cover layer 26. Thereby, a simple, cost-effective construction can be achieved. The cooling pad can be designed as one piece. Optionally, the side surfaces may also have common side layers 28, 29.

In the area of the first heat exchanger, the fluid channels 23 can advantageously have a cross-section with a flattened side, for example a semicircular cross-section as shown in FIG. 6. The flattened side is advantageously arranged facing a side or surface of the heat exchanger through which heat is to be absorbed or dissipated. In the shown embodiment, the heat is absorbed or dissipated via the upper cover layer 25. Advantageously the upper cover layer 25 can be implemented as a thin, heat conductive and fluid-tight membrane. The top layer 25 can form a side wall of the fluid channels. This can facilitate heat transfer, as thermal resistance can be reduced. With respect to the lower cover layer 26, the intermediate layer 27 may optionally provide insulating effect. Advantageously, a semicircular cross-section of the fluid channels 23, with a planar surface facing upwards, covered by a thin cover sheet for contact with a body surface for good heat transfer, may thus be provided with an optional rear insulation layer. Another advantage of flattened fluid channels 23 is that the thickness of the heat exchanger can be reduced.

It is to be understood that the second heat exchanger can optionally be adapted accordingly. The second heat exchanger can also comprise fluid channels. The fluid channels may have a flattened side which may face the cooling element 63 during operation of the cooling device 60.

FIG. 7 shows a schematic figure of a cooling system 1 with a sectional view of a cooling device 60. In the embodiment shown in FIG. 7, the cooling device 63 comprises an electrically operated Peltier element 81, which can be mounted on a cover 62. Alternatively, the Peltier element can also be arranged below the receptacle as shown in FIG. 2. It is to be understood that several cooling elements can also be provided. The cover 62 is arranged at a base body of the housing 65 by means of a hinge 82 and forms together therewith a receptacle 61 for the second heat exchanger 30.

The electrically operated Peltier element 81 cools, via the second heat exchanger 30, the heat transfer fluid 11 circulated by the pump device 50 in the hermetically sealed fluid circuit of the cooling pad 10. The excess heat, i.e. the heat extracted from the heat transfer fluid plus the electrical power of the Peltier element 81 can be dissipated via a heat sink 83 and optionally a fan 84.

Via the closed fluid circuit, the heat transfer fluid 11, now cooled down to a predetermined temperature, is transported to the first heat exchanger 20, which rests on a skin area 101, where it absorbs heat so that the skin area 101 is cooled as desired.

The cross-sections of the ducts can advantageously be optimized in such a way that only a minimum fluid volume is necessary, so that the thermal inertia of the system is minimized, but at the same time a sufficiently high flow rate can be realized and the energy expenditure for the circulation of the cooling medium does not become too high. Typical cooling capacities (heat dissipation from the tissue) are in the range of 20 to 60 mW/cm2. Typical cooling areas are in the range of 100 to 500 cm2. This results in cooling capacities in the range of 2 W to 30 W. In particular considering the usually time-limited thermotherapy, this is also possible with battery-powered devices. The pumping power for circulation may be 1 to 3 W or less.

The cooling device 60 can optionally comprise an energy storage 85 for mains-independent operation. Advantageously, the cooling device 60 also comprises a control device. The control device can implement the functions of a controller 86 and/or a monitoring device 89 as described above. For example, the control device can be implemented in the form of a microcontroller which, for example, controls a power controller for the power supply of the Peltier element and/or controls the drive 64. The cooling device 60 can optionally comprise a first and/or second temperature sensor 87, 88. In particular, a first temperature sensor 87 upstream of the cooling element 63 for measuring a return temperature and a second temperature sensor 88 downstream of the cooling element for measuring a flow temperature may be provided. Based on the temperature difference and a volume flow of the heat transfer fluid, a cooling capacity removed from the skin area 101 can be estimated.

As indicated in FIG. 7 and exemplarily shown below in FIGS. 8, 9 and 10, the pump device 50 can be adapted to be driven by a drive 64 of the cooling device 60 without any opening or lead-through. The drive 64 may comprise an electric motor 91 and a driver or carrier 92. The electric motor 91 can in turn be controlled via the control device 86. However, it is also possible to drive the pump device by means of an eddy-current drive without lead-through.

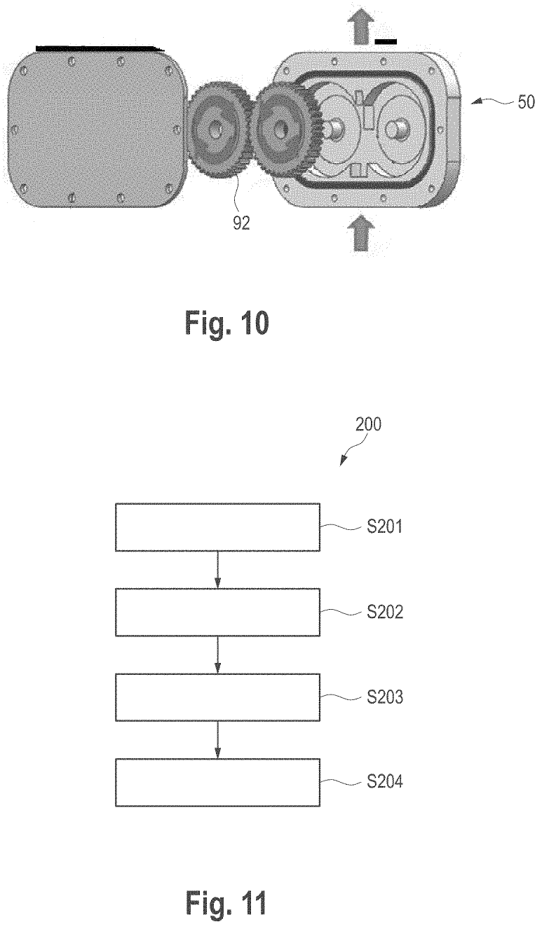

FIG. 8 shows a top view of the drive 64 with the electric motor 91 and the driver or carrier 92, which may comprise one or more magnets 93. With the magnets 93 a force can be transmitted to the pump device 50 without any lead-through. For example as shown in FIGS. 9 and 10, the pump device 50 can be implemented as magnetically coupled gear type pump. For this, one or more magnets 96 are provided on at least one gear wheel 95. If the cooling pad with the pump device 50 is now inserted into the cooling device 60 in such a way that the magnets 96 of the gearwheel 95 of the pump device 50 are placed over the magnets 93 of the drive unit 64, a force of the drive can be transmitted without opening or lead-through. Hence, the hermetically sealed fluid circuit can be maintained.

FIG. 11 shows a flow chart of a method 200 of operating a cooling pad and a cooling device. In a first step S201 the cooling pad and the corresponding cooling device are provided. In a second step S202 the second heat exchanger of the cooling pad is placed in a receptacle of the cooling device. In a third step S203 the first heat exchanger of the cooling pad is applied to a location to be cooled. In a fourth step S204 the cooling process with the cooling device is started. It is to be understood that cooling can also be used for non-therapeutic purposes, for example for cosmetic purposes or relaxation.

The method described herein allows for very easy handling, even by less experienced users. The application of the cooling device in step S203 can take place, for example, with a conventional orthosis or bandage or similar. Thus, the proposed cooling pad can easily be used in a variety of different application scenarios.

The solutions described herein may help to prevent swelling, relieve pain and/or prevent tissue or nerve damage, especially after accidents, surgery or sports injuries. Another advantageous application is cooling, especially of extremities, during or after chemotherapy to avoid or reduce side effects such as numbness, nerve and tissue damage, and hair loss. An advantageous application in veterinary medicine is the treatment of joint problems of horses. In this case, a battery-powered cooling device can be attached to the animal, for example by means of a carrying strap, and an advantageously flexible cooling pad adapted to the shape of the joint in question may provide optimum cooling.

1. A cooling pad for medical or veterinary applications,
the cooling pad comprising
a first heat exchanger;
a second heat exchanger,
a heat transfer fluid;
a piping system; and
an integrated pump device for the heat transfer fluid;
wherein the first heat exchanger and the second heat exchanger have the heat transfer fluid flowing through them;
wherein the piping system fluidically connects the first heat exchanger and the second heat exchanger;
wherein the pump device is adapted to cause an exchange of the heat transfer fluid between the first heat exchanger and the second heat exchanger;
wherein the cooling pad comprises a hermetically sealed fluid circuit for the heat transfer fluid, the fluid circuit comprising the first heat exchanger, the second heat exchanger, the piping system and the pump device.

2. The cooling pad according to claim 1, wherein the first or the second heat exchanger comprises a heat absorption area for absorbing heat and the other heat exchanger comprises a heat emission area for releasing heat.

3. The cooling pad according to claim 1, wherein the piping system comprises a flow line and a return line, wherein the flow line fluidly connects an inlet of the first heat exchanger and an outlet of the second heat exchanger; and wherein the return line fluidly connects an outlet of the first heat exchanger and an inlet of the second heat exchanger.

4. The cooling pad according to claim 1, wherein the pump device is adapted to be driven by an external drive without lead-through.

5. The cooling pad according to claim 4, wherein the pump device is adapted to be magnetically coupled to an external drive, in particular wherein the pump device is a magnetically coupled gear type pump.

6. The cooling pad according to claim 4, wherein the pump device is a peristaltic pump.

7. The cooling pad according to claim 1, wherein at least a portion of the first heat exchanger is mechanically flexible.

8. The cooling pad according to claim 1, wherein the first heat exchanger is adapted to a body part to be cooled.

9. The cooling pad according to claim 1, further comprising a diffusion-inhibiting coating.

10. The cooling pad according to claim 1, further comprising a fluid reservoir configured to compensate for fluid loss of the heat transfer fluid

11. The cooling pad according to claim 1, wherein first and/or second heat exchangers comprise cooling loops that are arranged bifilarly.

12. The cooling pad according to claim 1, wherein fluid channels in at least one of the first heat exchanger and the second heat exchanger have a semi-circular channel cross-section.

13. The cooling pad according to claim 1, wherein the first heat exchanger has a thickness between 1 mm and 10 mm, in particular between 2 mm and 8 mm, in particular between 4 mm and 6 mm.

14. The cooling pad according to claim 1, wherein the first heat exchanger comprises a heat exchanger surface and cooling loops arranged within the heat exchanger, and wherein the cooling loops are separated from the heat exchanger surface by a thin, thermally conductive and fluid-tight membrane.

15. The cooling pad according to claim 1, wherein the cooling pad comprises at least in parts a biocompatible, thermally conductive fabric layer, in particular in a region of a surface of the first heat exchanger.

16. The cooling pad according to claim 1, wherein fluid channels in at least one of the first heat exchanger, the second heat exchanger and the piping system have a diameter between 2 mm and 3 mm.

17. The cooling pad according to claim 1, wherein the piping system between the first heat exchanger and the second heat exchanger has a length between 2 cm and 300 cm, in particular between 5 cm and 100 cm, in particular between 10 cm and 50 cm.

18. The cooling pad according to claim 1, wherein the fluid circuit has a fluid volume of between 5 ml and 100 ml, in particular between 10 ml and 60 ml, in particular between 15 ml and 40 ml, in particular between 20 ml and 30 ml.

19. The cooling pad according to claim 1, wherein the heat transfer fluid comprises water and/or ethylene glycol.

20. The cooling pad according to claim 1, configured for a heat flow between 15 mW and 100 mW, in particular between 15 mW and 80 mW, in particular between 25 mW and 65 mW per square centimeter of surface area of the first heat exchanger.

21. The cooling pad according to claim 1, wherein the first heat exchanger and the piping system, and in particular also the second heat exchanger, have a common layer structure comprising
an upper cover layer,
a lower cover layer, and
an intermediate layer, in particular a foam layer, which is arranged between the upper cover layer and the lower cover layer; and
wherein fluid channels of the first heat exchanger and the piping system, and in particular also of the second heat exchanger, are arranged inside the intermediate layer.

22. The cooling pad according to claim 21, wherein the integrated pump device is disposed between the upper cover layer and the lower cover layer.

23. The cooling pad according to claim 1, further comprising
a receptacle adapted to accommodate the first heat exchanger or the second heat exchanger;
a cooling element, wherein the cooling element is adapted to supply heat to or remove heat from the first heat exchanger or the second heat exchanger when accommodated in the receptacle; and
a drive adapted to drive a pump device integrated within the cooling pad.

24. A cooling device according to claim 23, further comprising
a temperature sensor; and
a controller;
wherein the temperature sensor is configured to sense a temperature of a cooling pad placed in the receptacle;
wherein the controller is configured to control the drive device and/or the cooling element based on a temperature detected by the temperature sensor.

25. The cooling device according to claim 23, further comprising a monitoring device, wherein the monitoring device is adapted to compare a speed of the drive with a power consumption of the drive and to output an error message in case of an anomaly.

26. The cooling device according to claim 23, further comprising a monitoring device wherein the monitoring device is adapted to perform a coupling test of the drive unit of the cooling device with the pump device of the cooling pad.

27. The cooling device according to claim 23, further comprising a monitoring device wherein the monitoring device is configured to detect air bubbles in the heat transfer fluid of the cooling pad.

28. A cooling system for medical or veterinary applications, the cooling system comprising:
a cooling pad comprising
a first heat exchanger,
a second heat exchanger,
a piping system that fluidically connects the first heat exchanger and the second heat exchanger,
a heat transfer fluid flowing through the first heat exchanger, the second heat exchanger and the piping system, and
an integrated pump device for the heat transfer fluid, the pump device being adapted to cause an exchange of the heat transfer fluid between the first heat exchanger and the second heat exchanger, wherein the first heat exchanger, the second heat exchanger, the piping system and the pump device comprise a hermetically sealed fluid circuit for the heat transfer fluid; and
a cooling device comprising a receptacle and a cooling element, wherein the cooling element is adapted to supply heat to or remove heat from the first heat exchanger or the second heat exchanger when accommodated in the receptacle and a drive adapted to drive the pump device of the cooling pad.

29. A method of operating a cooling pad and a cooling device, the method comprising the steps of:
providing a cooling pad comprising
a first heat exchanger,
a second heat exchanger,
a piping system that fluidically connects the first heat exchanger and the second heat exchanger,
a heat transfer fluid flowing through the first heat exchanger, the second heat exchanger and the piping system, and
an integrated pump device for the heat transfer fluid, the pump device being adapted to cause an exchange of the heat transfer fluid between the first heat exchanger and the second heat exchanger, wherein the first heat exchanger, the second heat exchanger, the piping system and the pump device comprise a hermetically sealed fluid circuit for the heat transfer fluid,
providing a cooling device comprising a receptacle and a cooling element, wherein the cooling element is adapted to supply heat to or remove heat from the second heat exchanger of the cooling pad when the second heat exchanger of the cooling pad is placed in the receptacle and a drive adapted to drive the pump device of the cooling pad;
placing the second heat exchanger of the cooling pad into a receptacle of the cooling device;
application of the first heat exchanger of the cooling pad to a location to be cooled; and
starting the cooling process with the cooling device.
